(19) United States
(12) Patent Application Publication
(10) Pub. No.: US 2004/0260226 A1
(43) Pub. Date: Dec. 23, 2004

(54) Ankle strap

(76) Inventor: Robert Farrer Gilmour, Auckland (NZ)

Correspondence Address: YOUNG & THOMPSON, 745 SOUTH 23RD STREET, 2ND FLOOR, ARLINGTON, VA, 22202, US

(21) Appl. No.: 10/716,534

(22) Filed: Nov. 20, 2003

(30) Foreign Application Priority Data
Nov. 20, 2002 (NZ) 522707

(51) Int. Cl. A61F 13/00

(52) U.S. Cl. 602/65

(57) ABSTRACT

The invention consists in a foot and ankle support comprising a boot element having an upper part configured to contain a lower part of a user's leg and the use's ankle and a lower part configured to contain at least part of the user's foot. The boot element provides a front opening and a pair of side flaps defining the opening. Primary fastening means are provided operable to draw the side flaps together and each side flap has at least one aperture positioned to form a pair of adjacent apertures when the side flaps are pulled together. At least one first strap is provided able to engage with one side flap and be passed through the or an aperture in the other side flap and returned to the first side flap for engagement therewith, and secondary fastening means to further secure the boot element.

### FIELD OF INVENTION

This invention relates to a foot and ankle support particularly for human use.

### BACKGROUND OF INVENTION

Foot and ankle supports of this type are typically used to reduce the likelihood of injury to the foot and/or ankle or subtalar joint in conditions of high physical stress thereto. In addition or alternatively such supports are used to limit the local deformation of a foot or ankle which has already been injured, as a means of assisting the healing process.

It has been found that many of the known supports fail to achieve satisfactory results or impose unwanted constraints on desired movements of the foot or ankle. For example, if support straps are fastened to the support behind the ankle/subtalar joint axis, plantar flexion of the foot causes the straps to lose tension and therefore control of the foot and/or ankle or subtalar joints is reduced.

Our U.S. Pat. No. 5,899,872 describes and claims a construction which uses isometric straps in a substantially FIG. 8 arrangement about the boot part of a foot and ankle support. While this construction gives good results, there remains a need for a construction which has more flexibility in use, and which will resist movements that initiate inversion injury of the rear foot whilst walking.

### OBJECT OF THE INVENTION

It is therefore an object of the present invention to provide a foot and ankle support which will go at least some way towards meeting the foregoing requirements in a simple yet effective manner or which will at least provide the public with a useful choice.

### STATEMENT OF THE INVENTION

Accordingly the invention consists in a foot and ankle support comprising a boot element having an upper part configured to contain a lower part of a user's leg and the user's ankle and a lower part configured to contain at least part of the user's foot, the boot element providing a front opening and a pair of side flaps defining the opening, primary fastening means operable to draw the side flaps together and each side flap having a least one aperture positioned to form a pair of adjacent apertures when the side flaps are pulled together, and at least one first strap able to engage with one side flap and be passed through the or an aperture in the other side flap and returned to the first side flap for engagement therewith, and secondary fastening means to further secure the boot element.

Preferably the side flaps have inner and outer surfaces, the first strap and the inner and outer surfaces having part fastening means thereon to enable the strap to be secured to the inner surface of one side flap, passed through the aperture in that side flap, looped through the aperture in the other side flap and be secured on the outer surface of the first mentioned side flap.

Preferably a pair of apertures are provided on each side flap, one aperture being at or adjacent the upper part of the boot element and one aperture being at or adjacent the lower part of the boot element.

Preferably the inner and outer surfaces of each side flap have the pile thereon of a pile and hook-fastening device, the hooks being provided on the strap.

Preferably the secondary fastening means comprise two straps having a first end and a second free end, the first ends being affixed or affixable to the upper part of the boot element and each of said straps being affixed to extend forwardly and down and having a length to pass across the top of the foot, under the metatarsal region of the foot and diagonally back up to be fastened to the boot element, part fastening means being provided on the boot element and the straps.

Preferably the first strap or straps can be positioned at a selected angle to the flap.

To those skilled in the art to which the invention relates, many changes in construction and widely differing embodiments and applications of the invention will suggest themselves without departing from the scope of the invention as defined in the appended claims. The disclosures and the description herein are purely illustrative and are not intended to be in any sense limiting.

### BRIEF DESCRIPTION OF THE DRAWINGS

One preferred form of the invention will now be described with reference to the accompanying drawings in which:

FIG. 1 is a diagrammatic side elevation of a foot and ankle support according to one preferred form of the invention,

FIG. 2 is a diagrammatic front elevation of part of a foot and ankle support as shown in FIG. 1, and

FIG. 3 is a diagrammatic perspective view of a foot and ankle support according to a preferred form of the invention in use.

### DESCRIPTION OF THE PREFERRED EMBODIMENT

Referring to the drawings, a foot and ankle support is provided which comprises a boot element 1 having an upper part 2 and a lower part 3. The upper part 2 is designed to contain the lower part 4 of a user's leg in use and the lower part 3 is constructed to contain at least part of the foot 5 of a user in use. In particular, the upper part 2 contains a user's ankle.

The boot element 1 provides a central opening 10 between a pair of side flaps 11 and 12. The side flaps may cover the edges of a tongue 13 which is able to be placed behind the side flaps 11 and 12 and which may be connected to the side flaps, for example, by stitching at points 14 and 15. The tongue 13 is desirably of a flexible material.

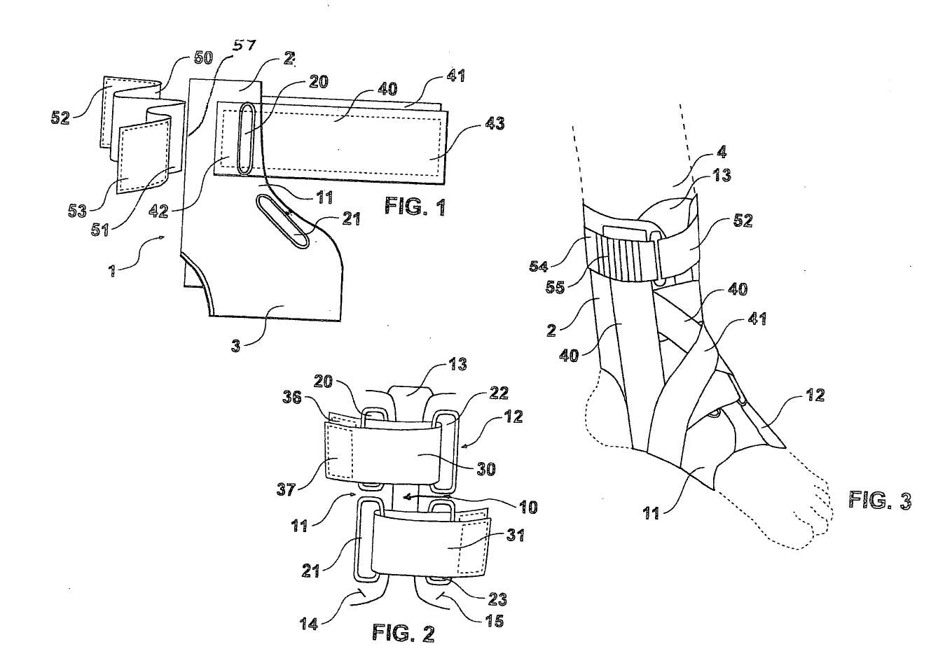

Primary fastening means are provided operable to draw the side flaps 11 and 12 together. To this end, each side flap includes at least one opening and in the preferred form each side flap provides a pair of openings. Openings 20 and 21 therefore may be provided on side flap 11 and openings 22 and 23 may be provided on the side flap 12. The primary fastening means also includes a pair of straps 30 and 31, which contain part-fastening means over at least some of their faces. The straps 30 and 31 are able to be fastened to both the inner and outer surfaces of the side flaps 11 and 12. This may be achieved by providing the inner and outer surfaces of the side flaps 11 and 12 with, for example, the pile of a hook and pile type connector and providing the straps 30 and 31 with the co-operating part of the hook and pile type fastener. Thus the side flaps may be provided with the pile over the whole of their extent on the inner and outer surfaces or the pile may be provided just in the areas which will need to be connected to the straps. The Straps therefore would then contain the hook parts of the hook and pile connectors at least in the areas 36 and 37 which are shown pecked in FIGS. 1 and 2. The intended use therefore is that a strap 30 or 31 would be connected to the inner surface of one side flap, for example, the inner surface of side flap 11 passed through the aperture 20, and looped through the aperture 22, for example, from the inner surface of side flap 12 to the outer surface. The strap 30 or 31 is then returned so that at least the area 37 of the strap may be secured to the outer surface of the side flap from which the strap originally extended.

In FIG. 2 the straps 30 and 31 are shown one extending from left to right then right to left and the other extending from right to left then left to right. This is for illustrative purposes only and in general both straps would loop in the same direction.

The area of the various hook and pile type fasteners, or at least one of the hook or pile straps 30 and 31, need not be attached at directly 90° to the longitudinal axis of the apertures 20, 21, 22 and 23. Thus the straps can be set at an angle best able to allow the boot element 1 to comply with or conform to the foot shape of individual users. By repositioning the fasteners the length of the strap is also able to be changed to achieve the most desirable fit of the boot element to the user's foot. Thus the areas of the fastener are made larger than strictly necessary to achieve secure fastening. The precise areas needed can be empirically determined providing a large part of the inner and outer surfaces of the flaps with, for example, the pile will achieve this desired result.

Secondary fastening means are provided and these may comprise firstly, a pair of straps 40 and 41 which are affixed to the outer surface of the boot, for example, by co-operating hook and pile fasteners between the boot and the straps 40 and 41 and are desirably positioned at a point at or adjacent the ankle when a foot is contained within the boot element 1. The connectors also desirably at about the mid-point vertically of the ankle but at least in general should not be behind the ankle. The straps 40 and 41 are provided at each end with part hook and pile fastenings shown pecked at 42 and 43. The straps 40 and 41 in use extend firstly downwardly across the top of the foot part. The straps are then looped under the metatarsal part of the foot and extend upwardly to be fixed, for example, by the fastening part 43 towards the upper parts of the part 2 of the foot and ankle support.

A further fastening element may be provided, for example, in the form of a pair of straps 50 and 51 which contain thereon parts of a hook and pile fastener 52 and 53. The straps 51 and 52 may be passed around the leg as shown in FIG. 3 and fixed by co-operation of the pads 52 and 53 in the area 54 for example. The straps 50 and 51 therefore further secure the straps 40 and 41 to the boot element 1. The straps 50 and 51 could be formed by a single strap which may be attached to the boot, for example, at rear part 57, generally behind the user's ankle.

Thus in use the user's leg and foot are placed into the boot and ankle support 1. The straps 30 and 31 are engaged at one end with the inner surface of the side flaps 11 and 12, passed through an aperture such as aperture 20, looped through the aperture such as aperture 22, tightened and secured back onto the outer surface of the side flaps such as side flap 11. Generally the straps 30 and 31 would loop in the same direction.

The straps 40 and 41 may then be passed around the user's foot and secured as above described and the whole construction can then be secured by use of the straps 50 and 51 which are secured by fasteners such as hook and pile type fasteners, as shown at 55.

Thus it can be seen that foot and ankle support is provided which has the advantage that the support is simple, effective and quick to apply and also to remove. It is a particular advantage of the invention that the straps attaching the side flaps can pull in either direction. This has the advantage that the straps can be readily reversed, if required, should it be necessary to prevent the foot from twisting in the opposite direction. The straps can also be used to pull the foot into pronation and also there is resistance to movement which could lead to initial inversion injury of the hind or rear foot. Furthermore, if the foot is over pronated at the forefoot then the straps can be arranged to supemate the foot. The angle and length of the straps drawing the side flaps together can be simply adjusted to achieve a snug and comfortable fit.

There is also a commercial advantage in that the straps allow a boot and ankle support to fit both left and right feet and in case can alter the relationship between leg and foot. Accordingly, the number of stock units required to hold is substantially reduced.

Throughout the description and claims of this specification the word “comprise” and variations of that word, such as “comprises” and “comprising”, are not intended to exclude other additives, components, integers or steps.

What is claimed is:

1. A foot and ankle support comprising a boot element having an upper part configured to contain a lower part of a user's leg and the use's ankle and a lower part configured to contain at least part of the user's foot, the boot element providing a front opening and a pair of side flaps defining the opening, primary fastening means operable to draw the side flaps together and each side flap having at least one aperture positioned to form a pair of adjacent apertures when the side flaps are pulled together, and at least one first strap able to engage with one side flap and be passed through the or an aperture in the other side flap and returned to the first side flap for engagement therewith, and secondary fastening means to further secure the boot element.

2. A foot and ankle support as claimed in claim 1, wherein the side flaps have inner and outer surfaces, the first strap and the inner and outer surfaces having part fastening means thereon to enable the strap to be secured to the inner surface of one side flap, passed through the aperture in that side flap, looped through the aperture in the other side flap and be secured on the outer surface of the first mentioned side flap.

3. A foot and ankle support as claimed in claim 1, wherein a pair of apertures are provided on each side flap, one aperture being at or adjacent the upper part of the boot element and one aperture being at or adjacent the lower part of the boot element.

4. A foot and ankle support as claimed in claim 1, wherein the inner and outer surfaces of each side flap have the pile thereon of a pile and hook-fastening device, the hooks being provided on the strap.

5. A foot and ankle support as claimed in claim 1, wherein the secondary fastening means comprise two straps having a first end and a second free end, the first ends being affixed or affixable to the upper part of the boot element and each of said straps being affixed to extend forwardly and down and having a length to pass across the top of the foot, under the metatarsal region of the foot and diagonally back up to be fastened to the boot element, part fastening means being provided on the boot element and the straps.

6. A foot and ankle support as claimed in claim 1, wherein the first strap or straps can be positioned at a selected angle to the flap.
